(19) United States
(12) Patent Application Publication
(10) Pub. No.: US 2003/0113282 A1
(43) Pub. Date: Jun. 19, 2003

(54) Low residue anhydrous antiperspirant stick composition

(76) Inventor: Thitiwan Buranachokpaisan, Morristown, NJ (US)

Correspondence Address: CHURCH & DWIGHT CO., INC., 469 North Harrison Street, Princeton, NJ, 08543, US

(21) Appl. No.: 10/021,210

(22) Filed: Dec. 12, 2001

(51) Int. Cl. A61K 7/32

(52) U.S. Cl. 424/65

(57) ABSTRACT

Disclosed are antiperspirant stick compositions that exhibit substantially less visible residue (whitening) upon application to the skin or after drying and which exhibit improved aesthetics and superior cosmetic properties. The compositions include a non-liquid organic ester emollient with a melting point between about 25° C. and about 60° C. (preferably isostearyl behenate); and an inert polymeric material (preferably a low molecular weight polyethylene; in addition to (a) a volatile (for example, cyclomethicone), (b) a non-volatile liquid emollient; (c) a gelling agent (for example, stearyl alcohol and hydrogenated castor oil); (d) at least one active antiperspirant material (for example, particulate antiperspirant aluminum zirconium tetrachlorohydrex salts); and (e) optionally other antiperspirant stick compatible materials.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The invention relates to the field of suspensoid stick compositions containing powdered antiperspirant active. The invention further relates to such formulations without the use of water or emulsions.

### BACKGROUND OF THE INVENTION

Wax-based stick compositions, which contain a wax-type solidifying agent, are known. It is also known to incorporate an antiperspirant active material, such as aluminum-zirconium-glycine complexes, in such wax-based stick compositions, to provide an antiperspirant stick. However, application of such wax-based antiperspirant stick product to the skin frequently results in objectionable aesthetic characteristics (such as unsatisfactory glide on the skin); moreover, such products unsatisfactorily leave visible residue (white residue) on the skin after application and after drying.

One manner of obtaining a dry/non-sticky application is achieved by devising an anhydrous formulation. Unfortunately, combining this with the goal of obtaining a low visible residue has been quite challenging, and not quite achieved. One manner of reducing residue is to make a product that is relatively hard. However, this results in both low application rates (negatively impacting on effectiveness) and such products are less satisfactory in smoothness of application. Alternatively, the product can be made softer (to maintain effectiveness), but this results in less aesthetically pleasing results (increase in visible residue or “oily application”) and in loss of high temperature stability. These qualities appear to be moving in opposite directions so that improvement in one is detrimental to the other.

Water-insoluble, high refractive index, non-volatile liquid components or emollients have been used to mask the residue in low residue anhydrous stick formulations. Examples are disclosed in U.S. Pat. Nos. 5,169,626; 4,985,238; 6,048,518; 5,972,319; 5,833,964; 5,449,511; 5,302,381; and 5,254,332, all of which are incorporated herein in their entirety by reference.

U.S. Pat. No. 4,919,934 to Deckner, et al. discloses wax-based cosmetic stick compositions containing specific amounts of a wax-type solidifying agent and a polyalphaolefin, and preferably an active component such as a sunscreen agent, analgesic, antiperspirant or deodorant active. This patent discloses that the stick composition preferably also includes at least one emollient, selected from volatile and non-volatile silicone oils and non-polar fatty acid and fatty alcohol esters, and that compositions which contain an antiperspirant active and/or deodorant active also preferably include at least one emulsifier. The contents of U.S. Pat. No. 4,919,934 is incorporated herein by reference in its entirety.

Various emollients have been suggested for use in cosmetic sticks such as antiperspirant/deodorant products. Some of these are described in U.S. Pat. No. 4,202,879 to Shelton and U.S. Pat. No. 4,725,432 to May, both of which are incorporated herein by reference. Examples of such emollients include fatty acid and fatty alcohol esters and water insoluble ethers

There have been attempts to provide low-residue antiperspirant solid sticks. See, for example, U.S. Pat. No. 4,822,603 to Farris, et al.; U.S. Pat. No. 5,254,332 to Greczyn, et al.; and U.S. Pat. No. 5,302,381 to Greczyn, et al., all of which are incorporated herein by reference. Each of U.S. Pat. No. 4,985,238 to Tanner and U.S. Pat. No. 5,169,626 to Tanner (each incorporated herein by reference) discloses low residue antiperspirant sticks containing specific amounts of a volatile silicone material; a particulate antiperspirant active; a low melting point wax; and a non-volatile paraffinic hydrocarbon fluid selected from mineral oils, branched-chain hydrocarbons containing an average of from about 16 to about 68 carbon atoms, and mixtures thereof. Non-essential components that can also be incorporated in the sticks include, for example, emollients, colorants, perfumes, and emulsifiers.

U.S. Pat. No. 5,225,188 to Abrutyn, et al. (incorporated herein by reference) discloses underarm formulations which contain volatile and/or non-volatile alkylmethylsiloxanes having a specific structure, which formulations may contain other components such as astringent antiperspirant compounds, suspending agents, conventional waxes, emollients, perfumes, coloring agents and other ingredients normally used in making underarm products. Incorporation of the alkylmethylsiloxanes in underarm formulations provide characteristics such as modified hardness, reduced whitening, improved feel, compatibility of ingredients, and control of vapor pressure.

It has also been proposed to incorporate phenyltrimethicone in antiperspirant formulations containing cyclomethicone as a vehicle, stearyl alcohol and hydrogenated castor oil as gelling agents, PEG-8 distearate, and aluminum-zirconium-tetrachlorohydrex-Gly, the phenyltrimethicone acting as a masking ingredient for the antiperspirant active ingredient to avoid a visible residue of the antiperspirant active on the skin.

U.S. Pat. No. 5,449,511 to Coe, the contents of which are incorporated herein by reference in its entirety, discloses a non-aqueous antiperspirant product that includes a non-aqueous carrier vehicle; an antiperspirant active salt suspended in particle form in the carrier vehicle; and a non-volatile, water-soluble, liquid (at 25° C.) masking agent that interacts with the antiperspirant active to essentially eliminate discernible whitening without substantially inhibiting the antiperspirant activity of the salt when the product is applied to the skin. The masking agent can be a non-volatile aliphatic compound (such as alcohols, ethers, silanols, silyl ethers, siloxanes and silicones) which contains disubstituted oxygen functionalities. This patent discloses that the masking agent preferably is a water-soluble, liquid, non-volatile emollient material, which reduces whitening by interacting with the particulates to produce an optical effect that tends to reduce light scattering and apparent whiteness. Illustrative masking agents disclosed in U.S. Pat. No. 5,449,511(incorporated herein by reference) include PPG-10 butanediol and dimethicone copolyols. This patent discloses that, in addition, for solid products, gelling agents may be included, examples of suitable gelling agents including hydrogenated castor oil, and fatty alcohols such as stearyl alcohol, among others, as well as blends and combinations.

U.S. Pat. No. 5,531,986 to Shevade et al (incorporated herein by reference) describes a low residue antiperspirant solid stick containing volatile and non-volatile silicone materials, dimethicone copolyol and high-melting and low-melting point waxes.

There have also been efforts to develop cosmetic compositions with improved aesthetics. U.S. Pat. No. 5,082,652 to Mayfield et al (incorporated herein by reference) teaches the use of an oil absorbent particulate material to prevent dusting of liquid particles and the addition of a silicone polymer to prevent dusting by the oil absorbent particulate material.

U.S. Pat. No. 4,917,882 to Strobridge (incorporated herein by reference) discloses a gel-type sunscreen comprising polyethylene and an ester of benzoic acid and C₁₂-C₁₅ alcohols. The product is described as having a generally non-greasy feel and uses the benzoate ester to provide a translucent, anhydrous vehicle for the sunscreen.

The use of waxes with a smaller crystalline size than the commonly used wax stearyl alcohol results in a more translucent residue than the same formulation with stearyl alcohol. Such materials are disclosed in U.S. Pat. Nos. 5,750,096 and 5,846,520, each of which is incorporated herein by reference. Other anti-whitening agents are disclosed in U.S. Pat. Nos. 5,922,308 and 5,531,986, each of which is incorporated herein by reference.

Notwithstanding the foregoing, it is still desired to provide an antiperspirant stick composition that exhibits substantially less whitening (residue) upon application to the skin or after drying thereon, which has desired cosmetic properties and antiperspirant efficacy, which has improved feel to the skin (in particular, reduced oiliness), better glide upon application and less tack.

Another important aesthetic quality in antiperspirant products is fragrance or lack thereof. The antiperspirant formulation must have an aesthetically appealing aroma. A suitable antiperspirant product should not have any disagreeable odor. While some undesirable odors can be masked by fragrances, some fragrances are not suitable for particular products. Furthermore, many consumers want fragrance free products so as not to clash with perfume or cologne aromatic principles, or because of sensitivities to such fragrances.

### OBJECTS OF THE INVENTION

It is therefore an object of the invention to provide an antiperspirant stick formulation that is both anhydrous and low visible residue on application.

It is a further object of the invention to provide an antiperspirant stick product having a high product delivery.

It is another object of the invention to provide an antiperspirant stick formulation, which, in the absence of fragrance, does not have a disagreeable odor.

It is yet another object to provide an antiperspirant stick formulation that does not have an oily or greasy feel on application.

It is still another object of the invention to provide an antiperspirant stick formulation having good high temperature stability

Still other objects of the invention will be apparent to those of ordinary skill in the art.

### BRIEF SUMMARY OF THE INVENTION

These and other objects of the invention can be achieved by an antiperspirant suspensoid stick formulation comprising:

(a) from about 25% to about 55% of a volatile material;

(b) from about 5% to about 35% of non-volatile liquid emollient(s);

(c) from about 0.5% to about 15% of non-liquid organic ester emollient(s) having melting point(s) between about 25° C. to about 60° C.;

(d) from about 5% to about 20% of organic wax base(s) having low melting point(s) or high melting point(s) or mixtures thereof;

(e) from about 0.05% to about 5% of low molecular weight polyethylene; and

(f) from about 15% to about 30% of a particulate antiperspirant active agent.

The composition may optionally contain one or more materials in one or more of the following groups: inert filler(s); wash-off agent(s); particulate suspending agent(s); surfactants; and fragrance(s).

### BRIEF DESCRIPTION OF THE SEVERAL VIEWS OF THE DRAWING

Not Applicable

### DETAILED DESCRIPTION OF THE INVENTION

The present invention is an antiperspirant suspensoid stick formulation comprising:

(a) from about 25% to about 55%, preferably about 25% to about 40%, more preferably about 27.5% to about 35%, still more preferably about 30% to about 33%, even more preferably 30.4% to about 32.5% of a volatile material, most preferably about 30.4% of a volatile material;

(b) from about 5% to about 35%, preferably about 10% to about 30%, more preferably about 15% to about 27.5%, still more preferably about 20% to about 25%, even more preferably about 21 to about 23%, most preferably about 22% of non-volatile liquid emollient(s);

(c) from about 0.5% to about 15%, preferably about 1% to about 10%, more preferably about 1.5% to about 5%, still more preferably about 1.75% to about 2.5%, most preferably about 2% of non-liquid organic ester emollient(s) having melting point(s) between about 25° C. to about 60° C.;

(d) from about 5% to about 20%, preferably about 10% to about 19%, more preferably about 15% to about 18%, most preferably about 16.5% of organic wax base(s) having low melting point(s) or high melting point(s) or mixtures thereof;

(e) from about 0.05% to about 5%, preferably about 0.1% to about 4%, more preferably about 0.5% to about 3%, still more preferably about 0.75% to about 2%, most preferably about 1% of low molecular weight polyethylene homo- or co-polymer; preferably having both a weight average molecular weight (M_{w}) and a number average molecular weight (Mₙ) independently of not more than about 3000, preferably not more than about 1000, more preferably not more than about 750, still more preferably about 250 to about 600, most preferably about 400 to about 500 and a ratio of M_{w}/Mₙ of about 0.9 to about 1.3, preferably about 1.0 to about 1.2, more preferably about 1.1; and

(f) from about 15% to about 30%, preferably about 17.5% to about 27.5%, more preferably about 20% to about 25%, most preferably about 22% of a particulate antiperspirant active agent.

The composition may optionally contain one or more materials in one or more of the following groups: inert filler(s), wash-off agent(s); particulate suspending agent(s); gelling agent(s); and fragrance(s).

The volatile material component (a) is typically selected from volatile liquid silicones, i.e. liquid polyorganosiloxanes, although non-silicone volatile materials may also be used. Generally, to be “volatile”, the material should have a measurable vapor pressure at 20° or 25° C. Generally, the vapor pressure of a volatile silicone is in the range of from about 1 or 10 Pa to 2 kPa at 25° C.

The volatile polyorganosiloxanes generally suitable for component (a) can be linear or cyclic or mixtures thereof. Preferred cyclic siloxanes include polydimethylsiloxanes preferably having about 3 to about 9 silicon atoms, preferably not more than about 7 silicon atoms, but especially preferably about 4 to about 6 silicon atoms, generally referred to as “cyclomethicones”. Cyclopentasiloxane is particularly preferred. Preferred linear siloxanes includes include polydimethylsilxoanes having about 3 to about 9 silicon atoms. These materials may be unsubstituted or substituted by one or more trimethylsiloxy groups resulting in branched (linear or cyclic) siloxanes as the case may be. Commercially available silicone oils include, but are not limited to, oils having grade designations 200, 344, 345, 244, 245, and 246 from Dow Corning Corporation; Silicone 7207 and Silicone 7158 from Union Carbide Corporation; SF 1202 from General Electric; and SWS-03314 from SWS Silicones, Inc. The linear volatile silicones generally have viscosities of less than 5 centistokes at 25° C. while the cyclic volatile silicones generally have viscosities of less than about 10 centistokes at 25° C. A more detailed description of various volatile silicone oils can be found in Todd et al, “Volatile Silicone Fluids for Cosmetics,” Cosmetics & Toiletries, 91, pp 27-32 (1976), the disclosures of which is incorporated herein by reference.

The non-volatile liquid emollient component (b) is generally not a single compound, but usually a group of compounds, which may be added separately or a blend of one or more of the appropriate compounds. The various subcomponents of component (b) include:

i. a (branched or straight chain) C₆₋₁₀alkyl (branched or straight chain) C₆₋₁₀alkanoate, preferably (branched or straight chain) C₇₋₉alkyl (branched or straight chain) C₈₋₁₀alkanoate, more preferably (branched or straight chain) C₈alkyl (branched or straight chain) C₉alkanoate, most preferably octyl isononanoate.

ii. a RCOO(C₂₋₃H₄₋₆O)ₙOCR¹ compound where R and R¹ are each independently selected from the group consisting of C₁₃₋₂₁alkyl, preferably selected from the group consisting of C₁₅₋₁₉alkyl, more preferably C₁₇alkyl. Most preferably, R and R¹ are the same. Preferably, the RCOO(C₂₋₃H₄₋₆O)ₙOCR¹ compound is a RCOO(C₂H₄O)ₙOCR¹. n is an integer of from 6-10, preferably 8. Most preferably, this component is polyethyleneglycol-8 distearate (PEG-8 distearate).

iii. a RCO(C₂₋₃H₄₋₆O)ₙH compound where R is a straight or branched chain alkyl of from 2 to 6 carbons, preferably from 3-5 carbons, more preferably 4 carbons, most preferably butyl. Preferably, the RCO(C₂₋₃H₄₋₆O)ₙH compound is a RCO(C₃H₆O)ₙH. n is an integer of from 10 to 18, preferably from 12 to 16, more preferably 14. Most preferably, this component is polypropyleneglycol-14 butyl ether (PPG-14 butyl ether).

iv. an aryl methicone preferably an aryl trimethicone. The aryl group is preferably phenyl. Preferably this component is phenyl trimethicone.

The subcomponents b(i) through b(iv) are generally present in the ratios of b(i):b(ii):b(iii):b(iv) of about 5 to about 15:about 1 to about 3:about 2.5 to about 7.5:about 2.5 to about 7.5, preferably about 7.5-about 12.5:about 1.5 to about 2.5:about 3.75 to about 6.25, most preferably about 10:about 2:about 5:about 5. Component b is most preferably octyl isononanoate 10 parts, PEG-8 distearate 2 parts. PPG-14 butyl ether 5 parts, and phenyl trimethicone 5 parts.

Further emollients which maybe included in the present invention formulations in addition to or in place of one or more of the foregoing are selected from the group consisting of

(a) esters which are not otherwise classified as alkoxylated carboxylic acids, glyceryl esters, isethionates, lanolin derivatives, phosphorous compounds, sulfosuccinates or sulfuric acid esters;

(b) alkoxylated alcohols wherein the alcohol portion is selected from aliphatic alcohols having 2-18 carbons (for example, 2-10 carbons and, more particularly, from 2-6 carbons); and the alkylene oxide portion is selected from the group consisting of ethylene oxide, polyoxyethylene, and polyoxypropylene having a number of alkylene units from 2-53 (for example, 4-30 units and, more particularly, from 12 to 15 units). Examples of suitable non-volatile emollients include isostearyl isostearate, isostearyl palmitate, benzyl laurate, PEG 12 and especially alkyl benzoates such as C₁₂-C₁₅ linear alkyl benzoates. The non-volatile emollients can include mixtures. Examples of such mixtures include isostearyl isostearate and C₁₂-C₁₅ alkylbenzoate; and isostearyl benzoate and benzyl laurate.

The non-liquid organic ester emollient(s) for component (c) is selected from R²O(O)CR³ compounds, in which R² is (branched or straight chain) C₁₄₋₂₂alkyl, preferably (branched or straight chain) C₁₆₋₂₀alkyl, more preferably (branched or straight chain) C₁₈alkyl, even more preferably branched, most preferably isostearyl, and in which R³ is (branched or straight chain, unsubstituted or substituted with hydroxy) C₂₋₂₅(alkyl or alkenyl), preferably (branched or straight chain) C₁₉₋₂₃alkyl, more preferably (branched or straight chain) C₂₁alkyl, even more preferably straight chain, most preferably R³CO— is behenoyl. In addition, the non-liquid organic ester emollient for component (c) may also be selected from glyceryl triesters where the acyls have a total number of carbons of from 27 to 39 carbon atoms, preferably 30 to 36, more preferably 33 carbon atoms, wherein the acyl groups may be the same or different, but are preferably the same. Preferred compounds for component (c) include, but are not limited to, isostearyl behenate, octadecyl propanoate, arachidyl propionate, cetyl ricinoleate, and glyceryl triundecanoate, with the most preferred component (c) compound being isostearyl behenate. As stated above, the component (c) material has a melting point of about 25° C. to about 60° C. Preferably the melting point is about 26° C. to about 45° C. These materials typically have a semi-solid appearance at room temperature. Preferably they are not one-phase clear liquids at 25° C.

The high and low melting point waxes of component (d) may be selected from those known in the art, provided that the wax (which may also be a combination of two or more agents) is soluble in the volatile material and able to be gelled therefrom. For example, the composition is heated in order to dissolve the wax in the vehicle and gelling occurs upon cooling of the composition. In particular, the wax may be selected from the group consisting of high melting point waxes (including beeswax, montan, ozokerite, ceresin, paraffin, synthetic waxes, hydrogenated castor oil); low melting point waxes (including fatty alcohols containing from about 8-20 carbons); and silicone waxes. A more preferred group of waxes consists of a mixture of stearyl alcohol and hydrogenated castor oil. The wax is then combined with the volatile component to form the gelling composition.

For an overall discussion of such wax gelling agents, attention is directed to the solidifying agents described in U.S. Pat. No. 4,919,934 to Deckner et al, the contents of which is incorporated herein by reference in its entirety. Examples of such waxes include crystalline waxes, cetyl stearate, stearyl stearate, cetyl myristate, cetyl palmitate, stearoxydimethicone, and microcrystalline waxes.

Various combinations, blends and mixtures of different materials can be utilized as the wax according to the present invention. Examples of such blends or mixtures include stearyl alcohol and beeswax, stearyl alcohol and hydrogenated castor oil, or cetyl alcohol and hydrogenated castor oil.

The polyethylene homo- or co-polymer suitable for use in the present invention as component (e) is preferably selected from the group consisting of homopolymers of ethylene, copolymers of ethylene and propylene, copolymers of ethylene and maleic acid or maleic anhydride, oxidized polyethylenes, and ethoxylated polyethylenes. Homopolyethylenes are preferred, suitable examples of which include, but are not limited to, Polywax 500, Polywax 655, and Polywax 1000, each available from Petrolite. Ethylene copolymers with propylene are exemplified by those sold under the name Petreolite® by Petrolite. Ethylene copolymers with maleic anhydride are exemplified by those polymers sold under the name Ceramer® by Petrolite, while oxidized polyethylenes are exemplified by those polymers sold under the name Unilin® and Unicid® by Petrolite, and ethoxylated polyethylenes are exemplified by those polymers sold under the name Unithox® by Petrolite.

The particulate antiperspirant component (f) can be any conventional antiperspirant material, including (but not limited to) antiperspirant active metal salts such as aluminum-zirconium tri-, tetra- and penta-chlorohydrate glycine complexes, which are coordination complexes of aluminum-zirconium tri-, tetra- or pentachlorohydrate and glycine in which some of the water molecules normally coordinated to the metal have been displaced by the glycine. Illustrative antiperspirant active metal salts include aluminum-zirconium tetrachlorohydrex gly (for example, Reach AZP-908 and Reach 908-0, each manufactured by Reheis Inc., (Berkeley Heights, N.J.)). The present invention is not limited to use of aluminum-zirconium tetrachlorohydrex gly, and other antiperspirant active metal salts (such as aluminum chlorohydrate), and/or other antiperspirant active materials, can be utilized in the stick composition of the present invention. Other active agents may be included as desired so long as they do not react with or are incompatible with the other required ingredients or each other.

The composition according to the present invention optionally includes, in addition to the foregoing components, one or more of surface active agents (surfactants), inert fillers and/or other materials such as, for example, fragrance, bacteriostats, bactericides, coloring agents, thickeners, solubilizers, chelating agents, preservatives, antioxidants, and processing aids.

Surfactants may be included in the compositions of the invention. Illustratively such surfactants include alkanolamides (such as N-alkyl pyrrolidone), ethoxylated amides (for example, the polyethylene glycol amide of tallow acid that conforms generally to the formula RC(O)NH(CH₂CH₂O)ₙH where RCO represents the fatty acyl derived from tallow and n has an average value of 50 (also called PEG-50 tallow amide)); amine oxides (for example, cocamidopropylamine oxide); ethoxylated fatty acids (for example, the polyethylene glycol diester of stearic acid that conforms generally to the formula CH₃(CH₂)₁₆C(O)—(OCH₂CH₂)ₙOC(O)(CH₂)₁₆CH₃ where n=average of 8 (also called PEG-8 distearate)); ethoxylated glycerides (for example, a polyethylene glycol derivative of Castor Oil with an average of 4 moles of ethylene oxide (also called PEG-4 castor oil)); glycol esters (for example, propylene glycol ricinoleate); monoglycerides (for example, glycerol myristate); polyglyceryl esters (for example, polyglyceryl-4 oleyl ether); polyhydric alcohol esters and ethers (for example, sucrose distearate); sorbitan/sorbitan esters (for example, sorbitan sesquiisostearate); triesters of phosphoric acid (for example, trioleth-8 phosphate (a material which is predominantly the triester of phosphoric acid and ethoxylated oleyl alcohol with an average of 8 moles of ethylene oxide)); ethoxylated lanolin (for example, a polyethylene glycol derivative of Lanolin with an average of 20 moles of ethylene oxide (also called PEG-20 lanolin)); propoxylated polyoxyethylene ethers (for example, the polyoxypropylene, polyoxyethylene ether of cetyl alcohol that conforms generally to the formula

CH₃(CH₂)₁₄CH₂(OCH(CH₃)CH₂)ₓ(OCH₂CH₂)_{y}OH

where x has an average value of 5 and y has an average value of 20 (also called PPG-5 ceteth-20)); and alkylpolyglycosides (for example, lauryl glucose). The surfactant (or surfactant blend) includes non-ionic compounds, and can also include blends thereof with cationic (for example, the polyethylene glycol amine of tallow acid that conforms generally to the formula RNH(CH₂CH₂O)ₙH (n=15, also called PEG-15 tallow amine)) or anionic (for example, sodium lauroyl laurate which is the sodium salt of the lauric acid ester of lauric acid) surfactants.

The surfactant or blend thereof incorporated in compositions according to the present invention can, illustratively, be included in amounts of about 0.2 to about 15%, preferably about 0.2 to about 5%, more preferably about 0.2 to about 2% and most preferably about 0.5 to about 1% by weight, of the total weight of the composition.

Inert fillers can be incorporated in the antiperspirant stick compositions of the present invention. Illustratively, the inert filler can be corn starch, talc, fumed silica and/or inorganic clays, or mixtures of these inert particulate materials. Colloidal fumed silica and talc, either separately or used in combination are preferred.

Various fragrances known in the art can also be incorporated in the antiperspirant solid stick composition of the present invention. These fragrances can be incorporated in amounts known in the art, for example, about 0.02 to about 3.0%, with scented products preferably having about 0.5 to about 3.0% by weight and unscented products typically having less than 0.5% by weight, of the total weight of the composition. When used in unscented products, fragrances are used as “masking agents” since the scents are not readily apparent but do cover or mask other undesirable aromas which may be inherent in the other components.

Known bacteriostats include bacteriostatic quaternary ammonium compounds such as 2-amino-2-methyl-1-propanol (AMP), cetyl-trimethylammonium bromide, cetyl pyridinium chloride, 2,4,4N-trichloro-2N-hydroxydiphenylether (Triclosan), N-(4-chlorophenyl)-N′-(3,4-dichlorophenyl)urea (Triclocarban) and various zinc salts. The bacteriostat can, illustratively, be included in the composition in an amount of 0.05-1.0% by weight, of the total weight of the composition.

The compositions of the invention are preferably prepared by combining all of the ingredients except for (a) the antiperspirant active; (b) talc (if present), and (c) fragrance (if present). The components are heated with stirring to about 90° C. until all of the waxes are completely melted. The antiperspirant active and talc are then added at a temperature of about 80° C. to about 90° C. Once the mixture has cooled to about 75° C., the fragrance is added. The mixture is allowed to cool to about 68° C. to about 72° C., at which time it is filled into appropriate containers and permitted to cool to room temperature.

### EXAMPLES

### Examples 1-3

Three formulations of the invention are prepared as set forth in the following

TABLE I

| Component | Formulation 1 | Formulation 2 | Formulation 3 |
|---|---|---|---|
| AlZr Tetrachlorohydrex Gly | 22% | 22% | 22% |
| Cyclopentasiloxane | 32.5% | 33% | 30.4% |
| Stearyl alcohol | 12% | 14% | 12.5% |
| Hydrogenated Castor Oil | 4% | 4% | 4% |
| Octyl Isononanoate | 10% | 10% | 10% |
| Isostearyl Behenate | 2% | 7% | 2% |
| Performalene 400 (Polyethylene, | 1% | 1.5% | 1% |
| New Phase Technologies) | | | |
| Talc | 3% | — | 4.5% |
| PEG-8 Distearate | 2% | 2% | 2% |
| PPG-14 Butyl Ether | 5% | — | 5% |
| Phenyl Trimethicone | 5% | 6% | 5% |
| Silica (Cab-O-Sil M5, Cabot) | 0.5% | 0.5% | 0.5% |
| PEG 25 propylene glycol stearate | — | — | 0.5% |
| Fragrance | — | — | 0.6% |
| Total | 100% | 100% | 100% |

The formulations are prepared as follows. All of the ingredients except for (a) the Al Zr Tetrachlorohydrex Gly, (b) the talc (if present), and fragrance (if present). The components are heated with stirring to about 90° C. until all of the waxes are completely melted. The Al Zr Tetrachlorohydrex Gly and talc are then added at a temperature of about 80° C. to about 90° C. Once the mixture has cooled to about 75° C., the fragrance is added. The mixture is allowed to cool to about 68° C. to about 72° C., at which time it is filled into appropriate containers and permitted to cool to room temperature.

Formulations 1 and 2 are tested for residue in a Minolta Chromameter (CR-310) in the L*a*b measuring mode. A thin sheet of foam (Foamtastic® FOM 302, distributed by Nicole™ of Mt Laurel, N.J.) is used as a substrate. The foam sheet is measured to obtain a baseline for L and L_{b} values. Each formulation is then applied onto the substrate as a thin film within the area of a 2 inch diameter circle to deposit 0.10 gram of the formulation. Measurements of L values are made at time=0, 3.5, and 24 hours from application and designated Lₜ. The same tests are conducted with three commercial products: Secret®, Degree®, and Lady's Choice Solid®. Whiteness, Wₜ is measured as Lₜ−L₀. Whiteness change with time (δWₜ) is expressed as Wₜ/W₀. Wₜ at t=0, 3.5, and 24 hours and δWₜ at t=3.5 and 24 hours is reported in Table 2 below.

TABLE 2

| | | | Secret (a | Degree (a | Ladys |
|---|---|---|---|---|---|
| | Formulation | Formulation | low residue | low residue | Choice |
| | 1 | 2 | product) | product) | Solid |
| W₀ | 3.6 | 2.9 | 1.9 | 3.8 | 4 |
| W_{3.5} | 1.9 | 1.3 | 2.2 | 7.2 | 10.7 |
| W₂₄ | 3.7 | 3.1 | 2.3 | 7.3 | 17.3 |
| δW_{3.5} | 0.53 | 0.45 | 1.16 | 1.89 | 2.68 |
| δW₂₄ | 1.03 | 1.07 | 1.21 | 1.92 | 4.33 |

I/We claim:

1. An antiperspirant cosmetic stick formulation comprising:
(a) from about 25% to about 55% of a volatile material;
(b) from about 5% to about 35% of non-volatile liquid emollient(s);
(c) from about 0.5% to about 15% of non-liquid organic ester emollient(s) having melting point(s) between about 25° C. to about 60° C.;
(d) from about 5% to about 20% of organic wax base(s) having low melting point(s) or high melting point(s) or mixtures thereof;
(e) from about 0.05% to about 5% of low molecular weight polyethylene homo- or co-polymer; and
(f) from about 15% to about 30% of a particulate antiperspirant active agent.

2. The formulation of claim 1 wherein said volatile material is a volatile silicone.

3. The formulation of claim 2 wherein said volatile silicone is a cyclomethicone.

4. The formulation of claim 3 wherein said cyclomethicone is predominantly cyclopentasiloxane.

5. The formulation of claim 1 wherein said non-volatile liquid emollient (b) is selected from:
i. a (branched or straight chain) C₆₋₁₀alkyl (branched or straight chain) C₆₋₁₀alkanoate;
ii. a RCOO(C₂₋₃H₄₋₆O)ₙOCR¹ compound where R and R¹ are each independently selected from the group consisting of C₁₃₋₂₁alkyl;
iii. a RCO(C2-3H4-6O)n H compound where R is a straight or branched chain alkyl of from 2 to 6 carbons;
iv. an aryl methicone and;
v. mixtures thereof.

6. The formulation of claim 5 wherein said non-volatile emollient (b) is selected from the group consisting of octyl isononanoate, polyethyleneglycol-8 distearate, polypropyleneglycol-14 butyl ether; and phenyl trimethicone.

7. The formulation of claim 5 wherein said non-volatile emollient (b) comprises at least octyl isononanoate, polyethyleneglycol-8 distearate, polypropyleneglycol-14 butyl ether; and phenyl trimethicone.

8. The formulation of claim 1 wherein said non-liquid organic ester emollient(s) (c) is selected from (a) R²O(O)CR³ compounds, in which R² is (branched or straight chain) C₁₄₋₂₂alkyl and (b) glyceryl triesters where the acyls have a total number of carbons of from 27 to 39 carbon atoms.

9. The formulation of claim 8 wherein said non-liquid organic ester emollient(s) (c) is selected from isostearyl behenate, octadecyl propanoate, arachidyl propionate, cetyl ricinoleate, and glyceryl triundecanoate.

10. The formulation of claim 1 wherein said high and low melting point waxes of component (d) are selected from (i) high melting point waxes selected from beeswax, montan, ozokerite, ceresin, paraffin, synthetic waxes, and hydrogenated castor oil; (ii) low melting point waxes selected from fatty alcohols.

11. The formulation of claim 10 wherein said high melting point wax is hydrogenated castor oil and said low melting point wax is stearyl alcohol.

12. The formulation of claim 1 wherein said low molecular weight polyethylene homo- or co-polymer (e) is selected from the group of homopolymers of ethylene, copolymers of ethylene and propylene, copolymers of ethylene and maleic acid or maleic anhydride, oxidized polyethylenes, and ethoxylated polyethylenes.

13. The formulation of claim 12 wherein said low molecular weight polyethylene homo- or co-polymer (e) is a polyethylene homopolymer.

14. The formulation of claim 12 wherein said low molecular weight polyethylene homo- or co-polymer (e) has a weight average molecular weight (M_{w}) and a number average molecular weight (Mₙ) independently of not more than about 1000, and a M_{w}/Mₙ of about 0.9 to about 1.3.

15. The formulation of claim 12 wherein said low molecular weight polyethylene homo- or co-polymer (e) has a weight average molecular weight (M_{w}) and a number average molecular weight (Mₙ) independently of about 400 to about 500, and a M_{w}/Mₙ of about 1.1

16. The formulation of claim 1 wherein said particulate antiperspirant component (f) is selected from aluminum-zirconium tri-, tetra- and penta-chlorohydrate glycine complexes, aluminum chlorohydrate, and aluminum chloride.

17. The formulation of claim 16 wherein said particulate antiperspirant component (f) is aluminum-zirconium tetrachlorohydrex gly.

18. The composition of claim 1 further comprising at least one material selected from surfactants, inert fillers, bacteriostats, and fragrances.

19. The composition of claim 18 wherein said inert fillers are selected from corn starch, talc, fumed silica, inorganic clays, and mixtures thereof.

20. An antiperspirant composition comprising
(a) about 25% to about 55% cyclopentasiloxane;
(b) about 5 to about 35% of non-volatile liquid emollients selected from octyl isononanoate, PEG-8 distearate, PPG-14 butyl ether, and phenyltrimethicone;
(c) about 0.5% to about 15% of isostearyl behenate;
(d) about 5% to about 20% of a mixture of hydrogenated castor oil and stearyl alcohol;
(e) about 0.05% to about 5% homopolyethylene having a weight average molecular weight (M_{w}) of about 400 to about 500, a number average molecular weight (Mₙ) of about 400 to about 500, and a ratio of M_{w}:Mₙ of about 1:1; and
(f) about 15% to about 30% of aluminum/zirconium tetrachlorohydrex gly.

21. An antiperspirant composition comprising
(a) about 30.4 to about 33% cyclopentasiloxane;
(b) (1) about 10% octyl isononanoate;
(2) about 2% PEG-8 distearate;
(3) 0% to about 5% PPG-14 butyl ether, and
(4) about 5% or about 6% phenyltrimethicone;
(c) about 0.5% to about 7% of isostearyl behenate;
(d) (1) about 4% hydrogenated castor oil; and
(2) about 12% to about 14% stearyl alcohol;
(e) about 1% to about 1.5% homopolyethylene having a weight average molecular weight (M_{w}) of about 400 to about 500, a number average molecular weight (Mₙ) of about 400 to about 500, and a ratio of M_{w}:Mₙ of about 1:1; and
(f) about 22% of aluminum/zirconium tetrachlorohydrex gly.

22. The antiperspirant of claim 21 further comprising:
(g) 0% to about 4.5% talc;
(h) about 0.5% silica;
(i) 0 to about 0.5% PEG-25 propylene glycol stearate; and
j) 0 to about 0.6% fragrance.

23. The antiperspirant of claim 22 which is selected from formulations 1, 2, and 3:

| Component | Formulation 1 | Formulation 2 | Formulation 3 |
|---|---|---|---|
| AlZr Tetrachlorohydrex Gly | 22% | 22% | 22% |
| Cyclopentasiloxane | 32.5% | 33% | 30.4% |
| Stearyl alcohol | 12% | 14% | 12.5% |
| Hydrogenated Castor Oil | 4% | 4% | 4% |
| Octyl Isononanoate | 10% | 10% | 10% |
| Isostearyl Behenate | 2% | 7% | 2% |
| Polyethylene | 1% | 1.5% | 1% |
| Talc | 3% | — | 4.5% |
| PEG-8 Distearate | 2% | 2% | 2% |
| PPG-14 Butyl Ether | 5% | — | 5% |
| Phenyl Trimethicone | 5% | 6% | 5% |
| Silica (Cab-O-Sil M5, Cabot) | 0.5% | 0.5% | 0.5% |
| PEG 25 propylene glycol stearate | — | — | 0.5% |
| Fragrance | — | — | 0.6% |
| Total | 100% | 100% | 100% |

24. A method of making an antiperspirant cosmetic stick formulation comprising:
(a) from about 25% to about 55% of a volatile material;
(b) from about 5% to about 35% of non-volatile liquid emollient(s);
(c) from about 0.5% to about 15% of non-liquid organic ester emollient(s) having melting point(s) between about 25° C. to about 60° C.;
(d) from about 5% to about 20% of organic wax base(s) having low melting point(s) or high melting point(s) or mixtures thereof;
(e) from about 0.05% to about 5% of low molecular weight polyethylene homo- or co-polymer; and
(f) from about 15% to about 30% of a particulate antiperspirant active agent; comprising
mixing, in any order, components (a) through (e) to form a mixture;
heating said mixture until all of said components (a) through (e) are in liquid form;
adding component (f) to said mixture in liquid form;
allowing the mixture to cool and filling the mixture into containers.
